(19) United States
(12) Patent Application Publication
(10) Pub. No.: US 2023/0221309 A1
(43) Pub. Date: Jul. 13, 2023

(54) METHOD, USE OF THE METHOD AND KIT FOR DETECTING BIOINDICATORS IN A SAMPLE

(71) Applicant: Forschungszentrum Juelich GmbH, Juelich (DE)

(72) Inventors: Oliver Bannach, Duesseldorf (DE); Andreas Kulawik, Erkrath (DE); Christian Zafiu, Vienna (AT); Dieter Willbold, Juelich (DE); Thi Tuyen Bujnicki, Juelich (DE)

(21) Appl. No.: 18/000,303

(22) PCT Filed: May 7, 2021
(86) PCT No.: PCT/DE2021/000084

§ 371 Date: Nov. 30, 2022

(30) Foreign Application Priority Data
Jun. 25, 2020 (DE) 10 2020 003 794.1

(51) Int. Cl. G01N 33/543

(52) U.S. Cl. CPC G01N 33/54346; G01N 33/54306; G01N 2470/04

(57) ABSTRACT

Provided is a method for the quantitative and/or qualitative determination of bioindicators, including the following steps: a) immobilizing capture molecules for the bioindicators on a substrate; b) bringing the bioindicators of a sample into contact with the capture molecules; c) immobilizing the bioindicators on the substrate by binding to capture molecules; d) bringing the bioindicators into contact with probes containing at least one detection molecule, and e) removing non-specifically bound molecules and particles; and f) binding the probes to the bioindicators, wherein the probes are capable of emitting a specific detection signal and steps b) and d) can take place simultaneously or d) before b), and wherein probes and capture molecules are used which have affine molecules or molecule parts that bind to at least one specific binding site of the bioindicators and these affine molecules or molecule parts of the probes and capture molecules do not overlap one another.

### CROSS-REFERENCE TO PRIOR APPLICATIONS

This application is a U.S. National Phase application under 35 U.S.C. § 371 of International Application No. PCT/DE2021/000084, filed on May 7, 2021, and claims benefit to German Patent Application No. 10 2020 003 794.1, filed on Jun. 25, 2020. The International Application was published in German on Dec. 30, 2021 as WO 2021/259406 A1 under PCT Article 21(2).

### FIELD

Embodiments of the invention relate to a method and a kit for detecting bioindicators in a sample, and to the use of the method for detecting a disease or checking the effectiveness of active substances and/or therapeutic methods.

### BACKGROUND

At present, bioindicators such as proteins, viruses, hormones, toxins or pesticides are detected using ELISA-like methods as standard. If the concentration of the bioindicator is particularly low, the so-called SIMOA method can be used.

### ELISA Method:

Using ELISA, proteins (e.g., antibodies) and viruses, but also low molecular weight compounds such as hormones, toxins and pesticides can be detected in a sample (blood serum, milk, urine, etc.). This makes use of the property of specific antibodies which bind to the substance (antigen) to be detected. An antibody is marked with an enzyme beforehand. The reaction catalyzed by the reporter enzyme serves to detect the presence of the antigen. The so-called substrate is converted by the enzyme, and the reaction product can usually be detected by color change, possibly also by chemiluminescence. The signal strength is a function of the antigen concentration that can be determined very precisely using a photometer, so that ELISA can be carried out for multiple measurements and can also be used for quantitative detection. Horseradish peroxidase (HRP), alkaline phosphatase (AP) or, more rarely, glucose oxidase (GOD) are usually used as reporter enzymes. In the case of alkaline phosphatase, for example, p-nitrophenyl phosphate (pNPP): is added as a dye substrate (synonym: chromogen), while o-phenylenediamine (oPD) is mostly used in the case of peroxidase. The alkaline phosphatase cleaves the phosphate residue from the colorless nitrophenyl phosphate and p-nitrophenol, which is pale yellow. The change in concentration of the dye formed by the enzymatic reaction can be monitored with a photometer according to the Beer-Lambert law. The intensity of the color increases with the concentration of the nitrophenol formed and thus also with the concentration of the antigen to be determined in the sample compared to a dilution series with known concentrations (standard series).

### Simoa Method:

The SIMOA Analyzer combines the single molecule analysis with digital ELISA display. Analytes are captured in solution by antibody-loaded beads instead of target-antibody interaction on an antibody-immobilized solid phase. Beads—whether target-loaded or not—are captured in femtoliter sized microcavities and provide a digital signal (on/off) for analysis.

A disadvantage of the methods according to the prior art is that, on the one hand, large amounts of sample are required in order to be able to carry out a detection. On the other hand, the methods have only a low dynamic measurement range. The previously known methods can only achieve maximum sensitivity in the picomolar range. SIMOA also has the disadvantage that special reagents (beads) are necessary for detection and technically complex equipment is required. Furthermore, with SIMOA only an indirect detection of the analyte through enzymatic secondary reactions is possible, which means that many influencing factors can lead to a falsification of the measurement results.

### SUMMARY

In an embodiment, the invention provides a method for the quantitative and/or qualitative determination of bioindicators, said method comprising the following steps: a) immobilizing capture molecules for the bioindicators on a substrate; b) bringing the bioindicators of a sample into contact with the capture molecules; c) immobilizing the bioindicators on the substrate by binding to capture molecules; d) bringing the bioindicators into contact with probes containing at least one detection molecule, and e) removing non-specifically bound molecules and particles; and f) binding the probes to the bioindicators, wherein the probes are capable of emitting a specific detection signal and steps b) and d) can take place simultaneously or d) before b), and wherein probes and capture molecules are used which have affine molecules or molecule parts that bind to at least one specific binding site of the bioindicators and these affine molecules or molecule parts of the probes and capture molecules do not overlap one another. Such a method is employed in additional embodiments to detect disease and to monitor therapies with bioindicators and/or check the effectiveness of active substances and/or therapeutic methods or determine if a person is to be included in a clinical study.

Another embodiment of the invention is a kit for carrying out the method for detecting bioindicators, wherein the kit comprises substrate, capture molecules; and probe molecules, optionally wherein Qdots are used instead of probe molecules, wherein the Qdots are coated with capture molecules having affine molecules or molecule parts which bind to at least one specific binding site of the bioindicators. A further embodiment of the Further embodiments of the invention relate to the use of the method for detecting a disease or for checking the effectiveness of active substances and/or therapeutic methods.

### BRIEF DESCRIPTION OF THE DRAWINGS

FIG. 1: Sample plate for simultaneous detection of different bioindicators;

FIG. 2: Detection of bioindicators with quantum dots (Qdots);

FIG. 3: Detection of bioindicators with quantum dots (Qdots) in conjunction with biotin/streptavidin;

FIG. 4: Detection of streptavidin in different concentrations; and

FIG. 5: Detection of a monomeric protein construct 2ALFA2GFP in different concentrations.

### DETAILED DESCRIPTION

An embodiment of the invention is a method for the quantitative and/or qualitative determination of bioindicators, said method comprising the following steps:
a) immobilizing capture molecules for the bioindicators on a substrate,
b) bringing the bioindicators of a sample into contact with the capture molecules,
c) immobilizing the bioindicators on the substrate by binding to capture molecules,
d) bringing the bioindicators into contact with probes containing at least one detection molecule, and
e) removing non-specifically bound molecules and particles, e. g. by washing,
f) binding the probes to the bioindicators,
   wherein the probes are capable of emitting a specific detection signal and steps b) and d) can take place simultaneously or d) before b), and wherein probes and capture molecules are used which have affine molecules or molecule parts that bind to at least one specific binding site of the bioindicators and these affine molecules or molecule parts of the probes and capture molecules do not overlap one another.

Steps c) and f) can also be carried out simultaneously in an advantageous manner.

In a further variant of the method, in which the bioindicators are brought into contact with the probes before they are brought into contact with the capture molecules, bioindicators marked with probes can be immobilized on the substrate.

Thus, the probes can be bound to the bioindicators before the bioindicators are brought into contact with the capture molecules and immobilized on the substrate.

Within the scope of the invention, the term “bioindicator” refers on the one hand to substances and molecules whose concentration depends on the condition of an organism. The exceeding or undershooting of a concentration limit value of the bioindicator allows conclusions to be drawn about a change in the condition of the organism (e.g.: disease, recovery). Examples of substances and molecules can be cells, genes, gene products, proteins, hormones, DNA, RNA or enzymes. On the other hand, the term “bioindicator” also refers to a substance or a molecule that can change the condition of an organism in concentrations that exceed the limit value. Hormones can be given as an example in this regard.

Another class of bioindicators that describe the condition of an organism are metabolites. These are proteins or small organic molecules that have already been modified by the body's own enzymes. The change in the metabolite can allow direct conclusions to be drawn about certain diseases.

In one embodiment of the invention, the method is characterized in that before step a) an immobilization of capture molecules for the bioindicators takes place on the substrate.

In a further embodiment of the invention and the method in accordance with the invention, the bioindicators can be immobilized on the substrate by being brought into contact with the capture molecules and by binding to the capture molecules.

In a further embodiment of the invention and the method in accordance with the invention, after bringing the bioindicators into contact with the probes, non-specifically bound molecules and particles can be removed, for example by washing.

In an embodiment, probes are selected which bind to the bioindicators, wherein the probes may, for example, also be capable of emitting a specific detection signal only after binding.

The bioindicators can be brought into contact with the capture molecules and the probes simultaneously.

The bioindicators can also be brought into contact with the probes prior to being brought into contact with the capture molecules.

In an embodiment of the invention, the method disclosed herein detects the bioindicators preferably in their monomeric form. The probes and/or capture molecules have affine molecules or molecule parts which recognize and bind to specific binding sites of the bioindicators. In order to be able to detect the bioindicators in their monomeric form, these affine molecules or molecule parts of the probes and capture molecules should bind to different specific binding sites of the bioindicators, so that the probes and capture molecules build up a directed specific bond to the bioindicators. For this purpose, these respective specific affine molecules or molecule parts of the probes and capture molecules should preferably not overlap. The binding site of bioindicators for the probes and the capture molecules should therefore differ in their peptide sequence by at least 5 amino acids.

The method disclosed herein makes it possible to detect the bioindicators in any sample and in a low concentration range of these bioindicators in a sample, preferably in a range which can be in the femtomolar or even in the sub-femtomolar range. Therefore, an individual detection can also be carried out without having to clean the sample in a complex manner.

In addition, the method can advantageously allow qualitative detection of bioindicators and/or also quantification and characterization in any sample. On the one hand, this advantageously ensures a direct and absolute quantification of the number of bioindicators, and on the other hand a characterization of the size distribution of bioindicators.

In an embodiment, the method is used to carry out a semi-quantitative detection of bioindicators if the concentration of the bioindicator is below or above a previously determined limit value compared to the reference state.

In certain embodiments, the bioindicators are detected in simple steps directly on any sample. The term “any sample” also means buffers with different additives or culture media. Alternatively, the sample can be taken ex vivo from body fluids or be a body fluid. Samples from the environment, such as water, plant and soil samples, as well as food, can be examined directly and the bioindicators can be detected.

In a further embodiment of the method, the bioindicators can also be immobilized directly on the substrate without capture molecules. In this variant of the method, the sample should first be pretreated with the bioindicators so that the biomarker to be examined has been separated from the sample, for example by immunoprecipitation, and is present homogeneously.

In a further possible embodiment of the method, the sample with the bioindicators is chemically fixed, for example with formaldehyde, after the bioindicators have been brought into contact with the probes.

Optionally, the sample may additionally be admixed with DNA and RNA binding probes after or during or prior to binding of the probes to the bioindicators.

In one embodiment of the invention, a detergent can be used after the chemical fixation in order to render the membrane of a bioindicator permeable, if necessary, and thereby allow the probes to penetrate into the interior of the bioindicator, for example while the probes are binding to the bioindicators.

For the purposes of the present invention, “quantitative determination” first means determining the concentration of the bioindicators, and thus also determining their presence and/or absence.

Preferably, quantitative determination also means the selective quantification of certain types of bioindicators. Such quantification can be proven via the corresponding specific probes.

For the purposes of the present invention, “qualitative determination” means characterization of the bioindicators.

An embodiment of the method is characterized in that in each case the affine molecules or molecule parts of the probes and/or capture molecules bind to at least two or more specific binding sites of the bioindicators, wherein here too these respective specific affine molecules or molecule parts of the probes and capture molecules preferably do not overlap.

In a further embodiment of the method, these respective affine molecules or molecule parts of the probes and/or capture molecules can bind to at least two or more specific binding sites of the bioindicators, wherein these respective affine molecules or molecule parts of the probes and/or capture molecules can bind to at least two or more identical or different binding sites of a bioindicator of the same type and/or can bind to at least two or more different binding sites of at least two different bioindicators.

On the one hand, this has the advantageous effect that, in particular in the case of at least two or more identical binding sites on a bioindicator, the specificity and stability of the binding of the capture molecules and/or probes to the bioindicator is increased.

In the possible embodiment of at least two or more different binding sites which bind to at least two different bioindicators, the advantageous effect consists in particular in that, for example, two different bioindicators can also be detected simultaneously in one method step or measurement process. This particular detection is also referred to below as “multiplex” detection. The signals in the multiplex detection for the two different bioindicators can then be evaluated, for example, via two separate evaluation channels for the probe signals, in particular different fluorescence channels. However, it is also possible to detect the two bioindicators using a common evaluation signal if a cumulative value is desired or sufficient as the evaluation result for both bioindicators. This can be of interest, for example, if two bioindicators are to be detected that belong to a common category, such as a clinical picture in which these bioindicators occur together.

The bioindicators are marked with one or more probes useful and/or specific for detection. In this case, in one embodiment of the invention, probes can be used, for example, which comprise at least one specific embodiment of an affine molecule or molecule part, which can then recognize and bind to at least one type of specific binding site of a bioindicator, wherein these affine molecules or molecule parts should not overlap with the affine molecules or molecule parts of the capture molecules as already explained above.

In a further embodiment of the invention, probes can be used which comprise at least two or more identical or different embodiments of affine molecules or molecule parts, which recognize and bind to at least two different or identical binding sites of a bioindicator of one type.

The affine molecules or molecule parts of the probes, which each recognize and bind to a specific binding site of the bioindicator, can be, for example, monoclonal antibodies, Fab fragments, aptamers, dyes or a construct of a plurality of these.

The affine molecules or molecule parts of the probes, each comprising at least two different embodiments of an affine molecule or molecule part, which recognize and bind to at least two binding sites of a bioindicator or two different bioindicators, can be, for example, bispecific antibodies or aptamers.

Table 1 below lists, but is not limited to, some possible bioindicators with binding sites for probes and/or capture molecules. These bioindicators have different epitopes, for example, as binding sites. The table also lists some affine molecules or molecule parts, in particular antibodies, which can bind to the binding sites of the bioindicators, in particular epitopes. The data listed as well as data on bioindicators not listed here with their respective binding sites are known to a person skilled in the art from the literature or product information from manufacturers of, for example, antibodies.

TABLE 1

| | | | | | Source/ | Source/ |
|---|---|---|---|---|---|---|
| | | | Example 1 | Example 2 | Reference | Reference |
| Bioindicator | Epitope 1* | Epitope 2* | Antibody⁽¹⁾ | Antibody⁽¹⁾ | Antibody 1 | Antibody 2 |
| Alpha-synuclein | 103-108 | 124-134 | 4B12 | 4D6 | 1) | 1a) |
| ANG-2 | 38-41 | 21-40 | 26-2F | ab8452 | 2) | |
| Aβ40 | 1-10 | C-terminus | 6E10 | #9682 | 3) | 3a) |
| Aβ42 | 1-10 | C-terminus | 6E10 | D3E10 | 4) | 4a) |
| IL-2 | 1-30 | 116-122 | 19B11/beta | basiliximab | 5) | 5a) |
| P-tau 181 | 210-230 | containing | Tau-5 | AT270 | 6) | 6a) |
| | | P 181 | | | | |
| P-tau 231 | 210-230 | containing | Tau-5 | AT180 | 7) | 7a) |
| | | P 231 | | | | |
| Tau | 210-230 | N-terminal | Tau-5 | Tau 13 | 8) | 8a) |
| | | | | (B11E8) | | |
| TDP-43 | D 247 | 203-209 | | 6H6E12 | 9) | 9a) |
| | | | | (60019-2-lg) | | |
| TNFα | 33-44 | 81-88 | 10F10 | 3B10 | 10) | 10a) |

*Amino acid number based on the respective amino acid sequence of the bioindicator
Sources/references for Table 1:
1) https://www.biolegend.com/en-us/products/purified-anti-alpha-synuclein--103-108-antibody-11222
1a) https://www.biolegend.com/en-us/products/purified-anti-alpha-synuclein-antibody-11220
2) https://absoluteantibody.com/product/anti-angiogenin-26-2f/Ab00400-1.4 Mouse IgG1/; https://pubmed.ncbi.nlm.nih.gov/7514035/
3) https://www.biolegend.com/en-us/products/purified-anti-beta-amyloid-1-16-antibody-11228
3a) https://www.biolegend.com/en-us/products/purified-anti-beta-amyloid--1-40-antibody-11230
4) https://www.biolegend.com/en-us/products/purified-anti-beta-amyloid-1-16-antibody-11228
4a) https://www.biolegend.com/en-us/products/purified-anti-beta-amyloid--1-42-antibody-11231
5) https://europepmc.org/article/med/8544854
5a) https://absoluteantibody.com/product/anti-il-2r-alpha-cd25-basiliximab/; https://pubmed.ncbi.nlm.nih.gov/17440057/
6) https://www.thermofisher.com/antibody/product/Tau-Antibody-clone-TAU-5-Monoclonal/AHB0042; https://pubmed.ncbi.nlm.nih.gov/17499212/
6a) https://www.thermofisher.com/antibody/product/Phospho-Tau-Thr181-Antibody-clone-AT270-Monoclonal/MN1050; https://pubmed.ncbi.nlm.nih.gov/7519852/
7) https://www.thermofisher.com/antibody/product/Tau-Antibody-clone-TAU-5-Monoclonal/AHB0042
7a) https://www.thermofisher.com/antibody/product/Phospho-Tau-Thr231-Antibody-clone-AT180-Monoclonal/MN1040; https://pubmed.ncbi.nlm.nih.gov/21871442/
8) https://www.thermofisher.com/antibody/product/Tau-Antibody-clone-TAU-5-Monoclonal/AHB0042
8a) https://www.biovision.com/tau-13-antibody-clone-b11e8.html; https://www.ncbi.nlm.nih.gov/pmc/articles/PMC4983528/
9) https://www.nature.com/articles/s41598-018-24463-3
9a) https://www.ptglab.com/Products/TARDBP-Antibody-60019-2-lg.htm; https://pubmed.ncbi.nlm.nih.gov/22133678/; https://www.scbt.com/p/tardbp-antibody-h-8
10) https://pubmed.ncbi.nlm.nih.gov/7590909/
10a) https://www.novusbio.com/products/cd30-tnfrsf8-antibody-3b10_nbp2-22206

Other possible bioindicators that can be detected using the method in accordance with the invention are listed below by way of example, but are not limited thereto. The binding sites of the bioindicators and the affine molecules or molecule parts of the probes and/or capture molecules suitable for this purpose can be determined and produced by methods known to a person skilled in the art.

### Bioindicators:

Angiogenesis Factor Panel 1 (ANG-2, FGFb, HB-EGF, HGF, PIGF, VEGF, VEGF-C, PDGFBB), BDNF, Biomarker Panel 1 (ICAM-1, MPO, NGAL, RANTES/CCL5, TIMP-1, VCAM-1), c-MET, C-Peptide, CA 19-9, CA-125, Cathepsin S, CCL-11/E-otaxin Assay Kit, CEA, Chemokine Panel (IP-10, ITAC, MCP-1, MIP-3b), Chemokine Panel 1 (rat) (MCP-1, MIP-1α, MIP-2, MIP-3α), CorPlex™ Cytokine Panel (IFN-γ, IL-1β, IL-4, 11-5, IL-6, IL-8, IL-10, IL-12p70, IL-22, TNF a), CRP, CXCL13, Cytokine 3-Plex A TNFα, IL-6, IL-10 (C3PA), Cytokine 3-Plex B TNFα, IL-6, IL-17A (C3PB), Cytokine Panel 1 (mouse) (IFN-γ, IL-1β, IL-2, IL-6, IL-10, IL-12p70, IL-17, TNF-α), Cytokine Panel 1 (non-human primate) (IFN-γ, IL-1β, IL-6, IL-8, TNF-α), Cytokine Panel 1 (rat) (IFN-γ, IL-1β, IL-2, IL-6, IL-10, KC, TNF-α), FGFb, G-CSF, GFAP, GM-CSF, GM-CSF (mouse), HB-EGF, HE4/WFDC2, HGF, HIV p24, ICAM-1, IFN-γ, IFN-γ (mouse), IFN-γ (non-human primate), IFN-γ (rat), IFNα, IL-10, IL-10 (mouse), IL-10 (rat), IL-12 p70, IL-12 p70 (mouse), IL-12p40/IL-23, IL-13, IL-15, IL-17A, IL-17A (mouse), IL-17A/F (mouse), IL-17C, IL-17F (mouse), IL-18, IL-1α, IL-1α (mouse), IL-1β, IL-1β (mouse), II-1β (non-human primate), IL-1β (rat), IL-2 (mouse), IL-2 (rat), IL-22 (mouse), IL-22 (total), IL-23, IL-23 (mouse), IL-28A, IL-3, IL-33, IL-36β, IL-4, IL-5, IL-6, IL-6 (mouse), IL-6 (non-human primate), IL-6 (rat), IL-7, IL-8, IL-8 (non-human primate), IP-10, ITAC, KC (rat), Leptin, LIF, MCP-1, MCP-1 (rat), MCP-3, MIP-1a, MIP-1β, MIP-2 (rat), MIP-3α (rat), MIP-3β, MMP Panel (MMP-3, MMP-9), MMP-3, MMP-9, MPO, Neuro 4-Plex B, Neurology 2-Plex A (Tau, Aβ42), Neurology 3-Plex A (Tau, Aβ42, Aβ40), Neurology 4-Plex A (NF-light®, Tau, GFAP*, UCHL-1*), NF-light®, NF-light® Advantage Kit (SR-X), NGAL, NSE, NT-proBNP, PD-1, PD-L1, Platelet-derived growth factor BB, PIGF, pNF-Heavy, PSA, RANTES/CCL5, Tau (mouse), TGFα, TGFβ, TIMP-1, TNFα (mouse), TNFα (non-human primate), TNFα (rat), TNFβ, TRAIL, Troponin-I, UCH-L1, VCAM-1, VEGF, VEGF-C.

As a result of the embodiment of the probes with at least two, for example identical, binding sites for the one bioindicator, a stronger and more specific binding of the probes to the bioindicator can be achieved, which then causes, for example, a higher stability of the binding compared to binding with only one binding site on the bioindicator, so that, for example, a subsequent stronger washing is possible, as well as a higher specificity of the detection of the bioindicator, since now at least two binding sites have to recognize and bind to the bioindicator. However, probes with at least two different binding sites for one bioindicator are also suitable.

In a further possible embodiment of the invention, a capture molecule is used which has at least two, preferably identical, specific binding sites for the bioindicator. As previously described for the probe with at least two identical binding sites, this more specific binding of the capture molecule to the bioindicator leads to a more stable and specific binding. For example, further method steps can then be carried out for the method, which steps can be carried out under less mild conditions for the bound molecules. Furthermore, the detection specificity is also increased here. However, capture molecules with at least two different binding sites for the one bioindicator are also suitable.

In a further embodiment of the invention, molecules which bind via at least two, preferably different, specific binding sites to the bioindicator can be used both as probes and as capture molecules. As a result of this embodiment, a further increase in the binding stability and specificity of the capture molecule and probe can be achieved with respect to a simple binding of the capture molecules and probes or also compared to a bispecific binding of probes or capture molecules with an identical binding site to the bioindicators.

In a further possible embodiment of the invention, the probe used has at least two different specific binding sites which can be assigned to at least two different bioindicators or can bind to at least two different bioindicators. With the aid of this probe, at least two different bioindicators can thus be detected simultaneously in one method step. The two different bioindicators are not detected separately from one another, but rather as a total value of both bioindicators. This is of interest, for example, when two bioindicators belonging to the same clinical picture are to be detected. In combination with these probes with the at least two different specific binding sites, for the at least two different bioindicators, capture molecules must then preferably be used, each of which has at least two different affine molecules or molecule parts for two different bioindicators, which can then bind to the at least two different bioindicators. As a result, at least two different bioindicators can be immobilized simultaneously on the substrate with a capture molecule and marked with the respective probes, which each specifically bind to one of the bioindicators, for example with a specific binding site, and can be detected in one method step.

As an alternative to the capture molecules, which can simultaneously bind two different bioindicators, two different capture molecules can also be used, which can then each specifically bind the different bioindicators.

In a further advantageous embodiment of the invention, both the probes and the capture molecules used have at least two different binding sites which can bind to at least two different bioindicators. Compared to an embodiment in which either the probes or the capture molecules have at least two different binding sites for at least two different bioindicators, the specificity of the detection of the bioindicators can be further increased by this embodiment.

The detection of at least two different bioindicators in one method step with probes and/or capture molecules which have at least two different binding sites which bind to at least two different bioindicators is referred to as multiplex detection, as already explained above.

In addition, the probes can advantageously contain at least one detection molecule or molecule part which is covalently bound to the affine molecule or molecule part for the bioindicators and can preferably be detected and measured by means of fluorescence measurement. These detection molecules, also referred to as direct or indirect reporter molecules, can comprise, for example, fluorochromes, quantum dots, or fluorescent proteins. The direct reporter molecule is directly bound to the specifically binding affine molecule or molecule part of the probe. Indirect reporter molecules have a high affinity for a binding site of a known molecule, which in turn is equipped with one or more fluorochromes. The resulting complex can be used as a probe. An example of a probe with an indirect reporter molecule can be biotin bound to an antibody-streptavidin molecule conjugated with fluorochrome. The group of indirect reporter molecules can comprise, for example, biotin-streptavidin, complementary DNA strands, DNA and DNA-binding proteins.

In one embodiment of the invention, the probes can have identical affine molecules or molecule parts with different detection molecules (or parts).

In another embodiment, different affine molecules or parts of molecules may be combined with different detection molecules or parts, or alternatively, different affine molecules or parts may be combined with identical detection molecules or parts.

According to another embodiment, mixtures of various probes are used.

The use of a plurality of different probes coupled to different detection molecules or molecule parts increases on the one hand the specificity of the signal (correlation signal), and on the other hand, this allows the identification of bioindicators which differ in one or more features. This can allow selective quantification and characterization of the bioindicators.

In a further embodiment of the present invention, the bioindicators can be detected using nanoparticles, in particular using quantum dots (Qdots). These quantum dots are coated for this purpose with a capture molecule for the bioindicator. As already described above, the capture molecules have affine molecules or molecule parts which bind to at least one specific binding site of the bioindicators. The coated quantum dots are introduced directly into the sample and specifically bind the bioindicators in the sample via the capture molecules. The advantage here is that the binding can take place very efficiently since it takes place in solution and can be promoted by mixing/stirring. After incubation, the sample/quantum medium mixture is applied to a substrate surface, for example a microtiter plate, the surface of which is likewise coated with a capture molecule, but which recognizes a different epitope on the bioindicator. Components not bound to the surface are removed by washing. Finally, the surface is microscopically examined and evaluated by fluorescence microscopy.

A further possible embodiment of the present invention includes the use of quantum dots coated with a capture antibody against the bioindicator. The coated quantum dots are added directly to the sample and bind the bioindicators in the sample. A second antibody is then added, which is marked with a biotin molecule, for example. The addition of quantum dots and biotinylated antibodies can also take place in reverse order. The advantage here is that the binding can take place very efficiently since it takes place in solution and can be promoted by mixing/stirring. After incubation, the mixture is applied to a microtiter plate, the glass bottom of which is coated with, for example, streptavidin. Components not bound to the surface can be removed by washing. Finally, the surface is microscopically examined and evaluated by fluorescence microscopy.

Due to the kinetic and steric conditions, the binding of the bioindicators to the Qdots in their monomeric form is favored over their oligomeric form, so that the bioindicators can preferably be detected in their monomeric form.

Quantum dots (Qdots) are commercially available on the market. Qdots are small nanoparticles with fluorescent properties. They can comprise nanoparticles with a core-shell material, for example CdSe/ZnS. These can be present, for example, in the range from 2-7 nm. With increasing diameter, the fluorescence of blue can be shifted in the direction of red.

When using Qdots in accordance with an embodiment of the invention, the additional use of probes can be dispensed with.

In a further embodiment in accordance with the invention, carboxy-functionalized Qdots can be used. For this purpose, for example, 0.1 nM Qdots (quantum dots) can be admixed with a thiol spacer acid mixture. This thiol spacer acid mixture consists, for example, of 550 mM tetramethylammonium hydroxide, 275 mM thiol spacer acid (e.g., 3-Mertopropropionic acid) in, for example, 1 ml CHCl₃, wherein the resulting aqueous phase is removed. This solution is mixed with the Qdots and 50 μL PBS added after 2 days. After shaking, the Qdots migrate into the aqueous phase and the desired carboxy-functionalized Qdots are obtained.

In a further embodiment, amino-terminated Qdots can be used. For this purpose, 1 mg dry Qdots are mixed with an amino spacer thiol mixture in methanol. This amino spacer thiol mixture consists of 100 mg amino spacer thiol (e.g.: 2,2′-diaminodiethyl disulfide). The mixture is sonicated until the solution absorbs the Qdots. These are separated by centrifugation and absorbed in water. Amino-terminated Qdots are in the aqueous phase.

In a further possible embodiment of the invention, monomers or epitopes, generally binding sites for the bioindicators, can be coupled to the nanoparticles. For this purpose, nanoparticles with carboxy termini which are converted into a reactive NHS ester can be used. The NHS ester is preferably formed with 1-Ethyl-3-(3-dimethylaminopropyl) carbodiimide (EDC) and/or N-hydroxysuccinimide (NHS). The NHS ester represents the reactive intermediate for reaction with a primary amine that is added as a monomer to the bioindicators to be detected.

A further variant of the method in accordance with the invention provides for amino-functionalized nanoparticles to react with a maleimide-spacer-carboxy, e.g., with 6-maleimido-caproic acid or maleimide-PEG (optional length)-COOH. This has the advantageous effect that a group which specifically reacts with thiols is introduced on the surface of the nanoparticle.

In a further possible embodiment, the aforementioned NHS ester particles are used to bind streptavidin to the surface of the nanoparticles.

The nanoparticles as such, i.e., without the binding site of the bioindicator, are novel and thus already achieve the object of the invention:

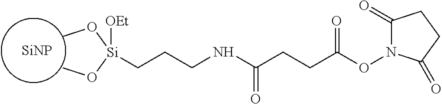 [SMILES: "SiNP" ; CCO[Si](CCCNC(=O)CCC(=O)ON1C(=O)CCC1=O)(OC)OC]

is a particularly preferred starting material with NHS esters on the surface of, for example, silica nanoparticles (SINP).

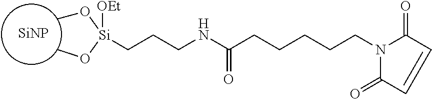 [SMILES: "SiNP" ; CCO[Si](CCCNC(=O)CCCCCN1C(=O)C=CC1=O)(OC)OC]

is a particularly preferred starting material with maleimide groups on the surface of, for example, silica nanoparticles (SINP).

A large number of the compounds depicted only once here is of course arranged on the surface of the Qdots. This number is advantageously determined precisely by calculation and depends on the diameter of the Qdot.

Nanoparticles with N-hydroxysuccinimide ester surfaces react with the primary amine of the monomer or a synthetic epitope of the bioindicators to be detected, possibly with elimination of N-hydroxysuccinimide.

This method is particularly suitable if the amino acid lysine is present one or more times in a peptide sequence of the monomer. This method is particularly suitable if the amino acid is not in the epitope region, i.e., the binding site for antibodies. If no lysine is present, either the N-terminus of the amino acid sequence itself can be used to bind to the Qdot, or the peptide sequences with additionally introduced lysines can be used.

Qdots with maleimide groups on the surface optionally react with existing or synthetically introduced thiols (e.g., cysteine) in the protein sequence of the monomer or epitope.

Qdots with a streptavidin surface bind particularly strongly biotinylated monomers or epitopes.

These silica Qdots react with the primary amine of the monomer of the bioindicator to be detected, possibly with the elimination of N-hydroxy-succinimide.

Modifications can be easily carried out due to the chemical surface properties. In addition, the material of the Qdots is stable in physiological buffer solutions and is considered to be harmless to health.

In particular, the biofunctionalized silica Qdots shown above represent stable, precisely sized standards with a precisely determinable number of accessible epitopes and are therefore also advantageously used as platform technology in diagnostic test procedures or in spiking experiments for the quantitative and qualitative determination of bioindicators.

In a preferred embodiment, the probe signal is determined in a spatially resolved manner, i.e., the signal emitted by the probe is detected in a spatially resolved manner. Accordingly, in this embodiment of the invention, methods based on a non-spatially resolved signal, such as ELISA or sandwich ELISA, are excluded. Compared to ELISA, the present method is around a thousand times more sensitive, since individual molecules can be counted (digital readout) and not a single integrated signal is generated from a bulk measurement.

A high spatial resolution is advantageous in the detection. In one embodiment of the method in accordance with the invention, so many data points are collected that they allow the detection of a bioindicator against a background signal which is caused, for example, by device-specific noise, other unspecific signals or non-specifically bound probes. In this way, as many values (read-out values) are read out as there are spatially resolved events, such as pixels. The spatial resolution determines each event against the respective background and thus represents an advantage over ELISA methods without a spatially resolved signal.

In one embodiment, the spatially resolved determination of the probe signal is based on total internal reflection fluorescence microscopy (tirfm) and the examination of a small volume element in the range from a few femtoliters to less than one femtoliter, compared to the volume of the sample of, for example, 10 to 1000 μl, or a volume range above the contact surface of the capture molecules with a height of 500 nm, preferably 300 nm, particularly preferred 250 nm, in particular 200 nm.

In an embodiment of the invention, bioindicators are detected which are selected from the group containing or consisting of cells, genes, gene products, proteins, hormones, RNA, and enzymes.

In one embodiment, the material of the substrate is selected from the group comprising or consisting of plastic, silicon and silicon dioxide. In a preferred alternative, glass is used as the substrate.

In a further embodiment of the invention, the capture molecules are covalently bound to the substrate.

In an alternative, a substrate having a hydrophilic surface can be used for this purpose. In an alternative, this can be achieved by the application of a hydrophilic layer, prior to step a), to the substrate. Consequently, the capture molecules bind, in particular covalently, to the substrate or to the hydrophilic layer with which the substrate is loaded.

The hydrophilic layer can be a biomolecule-repellent layer, so that the nonspecific binding of biomolecules to the substrate is advantageously minimized. The capture molecules can be immobilized, preferably covalently, onto this layer. These are affine with respect to a feature in the bioindicators. The capture molecules may all be identical, or mixtures of different capture molecules may be present.

Preferably, the capture molecules do not comprise a detection molecule or molecule parts suitable for detection.

In one embodiment, the hydrophilic layer can be selected from the group comprising or consisting of polyethylene glycol, poly lysine, preferably poly D lysine, and dextran or derivatives thereof, preferably carboxymethyl-dextran (CMD). Derivatives within the meaning of the invention are compounds which differ in some substituents from the parent compounds, wherein the substituents are inert compared to the method in accordance with the invention, i.e., do not produce a measurement signal which could lead to a falsification of the detection.

In another possible embodiment of the invention, the surface of the substrate can be functionalized before application of the hydrophilic layer by first hydroxylating the surface and then activating it with amino groups. In an alternative, this activation with amino groups can, for example, but not limited thereto, also take place by bringing the substrate into contact with APTES (3-aminopropyltrioxysilane) or with ethanolamine.

In order to prepare the substrate for the coating, one or more of the following steps can be carried out:
washing a substrate of glass or a glass carrier in an ultrasonic bath or plasma cleaner; alternatively, incubating in 5 M NaOH for at least 3 hours,
rinsing with water and subsequently drying under nitrogen,
dipping into a solution of concentrated sulfuric acid and hydrogen peroxide at a ratio of 3:1 for the activation of the hydroxyl groups,
rinsing with water to a neutral pH, subsequently washing with ethanol and drying under a nitrogen atmosphere,
dipping into a solution of 3-aminopropyltrietoxysilane (APTES) (1-7%), preferably in dry toluene or in a solution of ethanolamine,
rinsing with acetone or DMSO and water, and drying under a nitrogen atmosphere.

In a possible alternative embodiment, the substrate is brought into contact with APTES in the gas phase; the pretreated substrate, if necessary, is therefore vaporized with APTES.

For coating with dextran, preferably carboxymethyl-dextran (CMD), the substrate is incubated with an aqueous solution of CMD in a concentration of 10 mg/ml or 20 mg/ml and optionally N-ethyl-N-(3-dimethylaminopropyl)carbodiimide (EDC), (200 mM) and N-hydroxysuccinimide (NHS), (50 mM) and subsequently washed.

In one embodiment, the carboxymethyl-dextran can be covalently bonded to the glass surface, which was first hydroxylated and, in particular, functionalized with amino groups.

For example, microtiter plates, preferably with a glass bottom, can also be used as the substrate. Since the use of concentrated sulfuric acid is not possible when polystyrene frames are used, the glass surface is activated analogously in an embodiment variant of the invention.

Capture molecules which are affine with respect to a feature of the bioindicator to be detected are immobilized on this hydrophilic layer, preferably covalently. This feature may be a protein. The capture molecules may all be identical or be mixtures of various capture molecules.

In one embodiment of the present invention, the capture molecules, preferably antibodies, are immobilized on the substrate, optionally after activation of the CMD-coated carrier by a mixture of EDC/NHS (200 and 50 mM, respectively).

Remaining carboxylate end groups to which no capture molecules were bound can be deactivated. Ethanolamine is used to deactivate these carboxylate end groups on the CMD spacer. Prior to the application of the samples, the substrates or carriers are optionally rinsed with buffer.

The sample to be measured is brought into contact with the substrate prepared in this way and optionally incubated. For example, endogenous fluids or tissue can be used as the sample to be examined. In one embodiment of the present invention, the sample is selected from liquor (CSF), blood, plasma and urine. The samples may undergo various processing steps known to the person skilled in the art.

In one embodiment of the present invention, the sample can be applied directly to the substrate, e.g., the uncoated substrate, optionally by covalent bonding. If necessary, binding to an activated surface of the substrate takes place.

In a further embodiment of the present invention, the sample can be pretreated according to one or more of the following method steps:
diluting with water or buffer,
treatment with enzymes, for example proteases, nuclease, lipases,
centrifuging,
precipitation,
competition with probes to displace any antibodies present.

Non-specifically bound substances can be removed by washing steps.

In a further step, the immobilized bioindicators can be marked with one or more probes which serve for further detection. As described above, the individual steps can also be performed in a different order according to embodiments of the invention.

By suitable washing steps, excess probes which are not bound to the bioindicators are removed.

In an embodiment, these excess probes need not be removed. This eliminates one washing step, and there is no equilibrium shift towards dissociation of the bioindicator-probe complexes or compounds. Due to the spatially resolved detection, the excess probes are not recorded during the evaluation.

In one embodiment, the binding sites of the bioindicators are epitopes and the capture molecules and/or probes are antibodies and/or antibody parts and/or fragments thereof. In a further advantageous embodiment, probes which recognize at least two different epitopes on the bioindicator with increased avidity are used, wherein it is to be observed that they are not already occupied by the capture molecules. The specificity of the detection can be increased in an advantageous manner by using at least bispecific or more specific probes which recognize at least two or more than two epitopes on the bioindicator.

In a preferred embodiment of the present invention, the capture molecules and probes differ. For example, different antibodies and/or antibody parts and/or fragments can be used as capture molecules and as probes.

In a further embodiment of the present invention, different probes are used simultaneously.

In a further alternative of the present invention, at least two or more different capture molecules and/or probes are used which, for example, contain different antibodies and, if appropriate, also carry different fluorescent dye markings.

For detection purposes, the probes are characterized in that they emit an optically detectable signal selected from the group consisting of fluorescence, bioluminescence and chemiluminescence emission and absorption.

The probes are preferably marked with fluorescent dyes. The dyes known to a person skilled in the art can be used as fluorescent dye. Alternatively, GFP (Green Fluorescence Protein), conjugates and/or fusion proteins thereof, and quantum dots may be used.

For quality control of the surface, e.g., to prove the uniformity of the coating with capture molecules, capture molecules marked with fluorescent dyes can be used.

For this purpose, a dye is preferably used which does not interfere with the detection of the fluorescent dye of the probe on the bioindicator. This enables subsequent control of the structure and standardization of the measurement results.

The immobilized and marked bioindicators are detected by means of imaging the surface, e.g., using laser scanning microscopy. As high a spatial resolution as possible determines a high number of pixels, as a result of which the sensitivity and the selectivity of the method can be increased, since structural features can also be imaged and analyzed. Thus, the specific signal in front of the background signal (e.g., non-specifically bound probes) increases.

Detection preferably takes place, for example, with spatially resolving fluorescence microscopy by a TIRF microscope, as well as the corresponding superresolution variants thereof, such as, e.g., STORM, dSTORM.

In one embodiment of the present invention, a laser focus, such as is used in laser scanning microscopy, or an FCS (fluorescence correlation spectroscopy system) is used for this purpose as well as the corresponding super-resolution variants, such as STED, PALM or SIM. By highly sensitive fluorescence microscopy, all individual fluorescence-marked probes can now be detected on the surface and counted more or less individually. These appear in the image data as pixels with a gray level value above the background signal (digital read out). This also results in the amount of target molecules to be detected—in principle with single molecule sensitivity. In contrast to ELISA, this method results in as many read-out values as there are spatially resolved events (e.g., pixels). Depending on the number of different probes, this information is advantageously multiplied. This multiplication applies to each detection event and leads to an information gain since it discloses further properties, e.g., a second feature, via bioindicators. As a result of such a structure, the specificity of the signal can be increased for each event.

The probes can be selected such that the presence of individual bioindicators, such as individual membrane proteins, does not affect the measurement result.

The probes can be selected such that bioindicator species (phenotypes) can be determined for each individual bioindicator.

Additional probes can be selected to differentiate between DNA/RNA-containing bioindicators and thus provide information about the interior of the bioindicators. For example, fluorophores which bind DNA/RNA, such as DAPI from Hoechst, can be used for this purpose.

For evaluation, the spatially resolved information, e.g., the fluorescence intensity of all probes used and detected is used in order to determine, for example, the number of individual bioindicators, their size and their features.

For example, algorithms for background minimization and/or intensity threshold values can also be used for further analysis as well as pattern recognition.

Further image analysis options include, for example, the search for local intensity maxima in order to obtain from the image information the number of bioindicators detected and also to be able to determine the particle sizes.

In order to make the test results comparable with one another over distances, times and experiments, internal and/or external standards can be used.

Two possible embodiments of the method in accordance with the invention are described below by way of example:

a) Consecutive Method:

In this embodiment of the method, a plurality of different bioindicators is quantified simultaneously in a single sample.

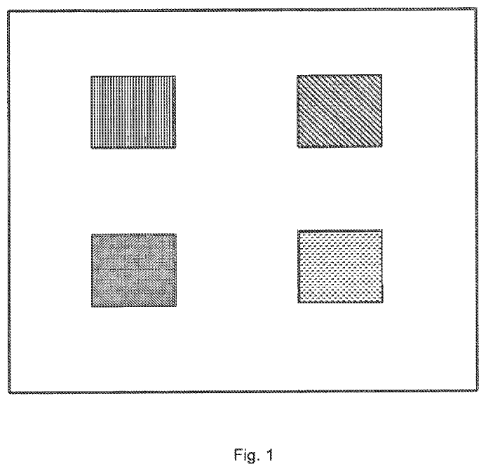

For this purpose, different capture molecules are each applied to different regions of the surface of the substrate, such as, for example, a glass or plastic plate with bores for receiving samples (spotted, see FIG. 1). The sample to be examined is then applied and the different bioindicators to be analyzed are immobilized on the respective regions with the aid of the capture molecules specific for the respective different bioindicators. Various probes are used for detection, which specifically recognize and specifically bind to the bioindicators to be analyzed. These probes can be marked with the same fluorochrome or with different fluorochromes. Finally, the respective regions of the sample plate are read out one after the other (bioindicator 1, 2, 3, 4, etc.) by fluorescence microscopy. The different bioindicators are then detected in a location-dependent manner in their respective color channels or are detected in a location-dependent manner in a color channel.

b) Simultaneous Method:

In this embodiment of the method, a plurality of different bioindicators is quantified simultaneously in a single sample.

For this purpose, a mixture of capture molecules is applied to the different regions of the surface of the substrate, such as a glass or plastic plate with bores for receiving samples. The sample to be examined with the different bioindicators is then applied, and the different bioindicators to be analyzed are immobilized on the surface with the aid of the specific capture molecules for the respective different bioindicators. Different probes that specifically bind to the bioindicators to be analyzed are used for detection. The different probes are each marked with different fluorochromes. Finally, the surface is measured by fluorescence microscopy; the different bioindicators can then be specifically detected in their respective color channels.

An embodiment of the present invention relates to a kit containing one or more of the following components:
substrate, optionally with hydrophilic surface,
capture molecule(s),
probe,
substrate with capture molecule,
solutions,
standard,
buffer.

The monomer of the bioindicator to be examined can preferably be used here as a standard, wherein this monomer is preferably obtained from an organism which was produced recombinantly or chemically synthesized. The standard must at least have all sequences or structures that are also recognized by the probes and capture molecules used and that have as similar an affinity as possible, in particular a similar KD value.

The compounds and/or components of the kit of the present invention may be packaged in containers, optionally with/in buffers and/or solution.

Alternatively, some components may be packaged in the same container. Additionally or alternatively, one or more of the components could be absorbed on a solid carrier, such as a glass plate, a chip or a nylon membrane, or on the well of a microtiter plate. The substrate then comprises such a microtiter plate.

Further, the kit may include instructions for use of the kit for any of the embodiments.

In a further embodiment of the kit, the capture molecules described above are already immobilized on the substrate. In addition, the kit may contain solutions and/or buffers. To protect the coating and/or the capture molecules immobilized thereon, they can be covered with a solution or a buffer.

A further embodiment of the present invention is the use of the method in accordance with the invention for the detection of bioindicators in any sample for the quantification and thus titer determination of bioindicators.

Advantageously, the method can also provide evidence of a disease, such as cardiovascular, kidney and cancer diseases, the detection of an immune response. The method can be used in substance development, for the direct and absolute quantification of bioindicators, with the diagnostics accompanying therapy (target engagement), differential diagnostics, detection of protein-protein interaction and/or typing of bioindicators.

A further embodiment of the present invention is the use of the method in accordance with the invention for monitoring therapies with bioindicators as well as for monitoring and/or checking the effectiveness of active substances and/or therapeutic methods. The method can therefore be used in clinical tests, studies as well as in therapy monitoring. For this purpose, samples are measured according to the method according to the invention and the results are compared.

A further embodiment of the present invention is the implementation of the method in accordance with the invention to determine the efficacy of active substances against diseased cells. The results are compared with one another on the basis of the characterization of bioindicators in samples. The samples are, accordingly, body fluids taken before, after, or at different times after the administration of the active substances or after the therapeutic method has been performed. According to embodiments of the invention, the results are compared with a control which was not subjected to the active ingredient and/or therapy. The results are used to select active ingredients and/or therapies.

A further embodiment of the present invention is the implementation of the method in accordance with the invention to determine if a person is to be included in a clinical study. For this purpose, samples are measured by the method according to the invention and the decision is made with respect to a limit value.

### Examples

The invention is explained herein with reference to figures and embodiments, without the invention thereby being limited to these figures or embodiments.

FIG. 1 shows a sample plate for the simultaneous detection of different bioindicators within one investigative approach.

FIG. 1 shows a sample plate for the simultaneous detection of different bioindicators within one investigative approach, also known as a multiplex method, in which the glass surface of the sample plate has four regions, in particular bores or depressions, each of which can be used as a reaction space for the method in accordance with the invention.

This method is described by way of example herein.

In the following embodiment of the method, a plurality of different bioindicators is quantified consecutively in a single sample in one investigative approach. For this purpose, different capture molecules are applied to different regions of the sample plate surface, also referred to as spotted. In FIG. 1, these are the four differently shaded square regions, in particular bores or depressions of the sample plate. The sample is then applied and the bioindicators to be analyzed are immobilized on the respective region with the aid of the specific capture molecules. Different probes that recognize the bioindicators to be analyzed are used for detection. These probes can be marked with the same fluorochrome or with different fluorochromes. Finally, the respective spots are read out one after the other (bioindicator 1, 2, 3, 4, etc.) by fluorescence microscopy. The different bioindicators are then detected in a location-dependent manner according to their respective color channels or are detected in a location-dependent manner in a color channel.

In a further embodiment of the method, a plurality of bioindicators is quantified simultaneously in a single sample. For this purpose, a mixture of capture molecules is applied to the designated regions/bores or depressions of the sample plate. The sample is then applied to these regions and the bioindicators to be analyzed are immobilized on the surface. Different probes that bind to the bioindicators to be analyzed are used for detection. The different probes are each marked with different fluorochromes. Finally, the surface is read out by fluorescence microscopy; the different bioindicators are then detected in their respective color channels.

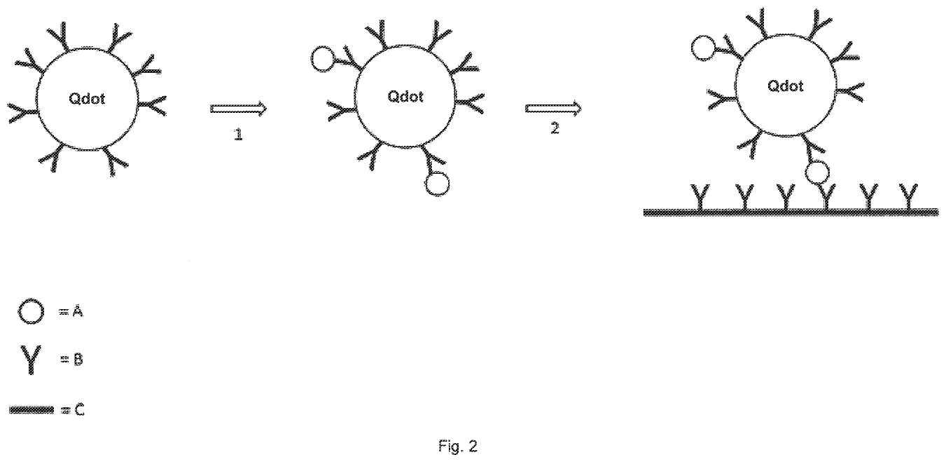

FIG. 2 shows a detection of bioindicators in accordance with embodiments of the invention using quantum dots (Qdot). The Qdots are first coated with a capture molecule (B) for the bioindicator (A). The Qdots coated with the capture molecules (B) are added directly to the sample in step 1 and specifically bind the bioindicator (A). After incubation, the bioindicator (B)/Qdot mixture is applied to the surface of a sample plate, for example a microtiter plate, in step 2. The surface of the sample plate (C) is coated with capture molecules (B), which, however, recognize a different binding site on the bioindicator (A). Components not bound to the surface of the sample plate (C) are removed by washing. The surface (C) is then evaluated by, for example, fluorescence microscopy, which detects the fluorescent Qdots bound to the surface of the sample plate (C).

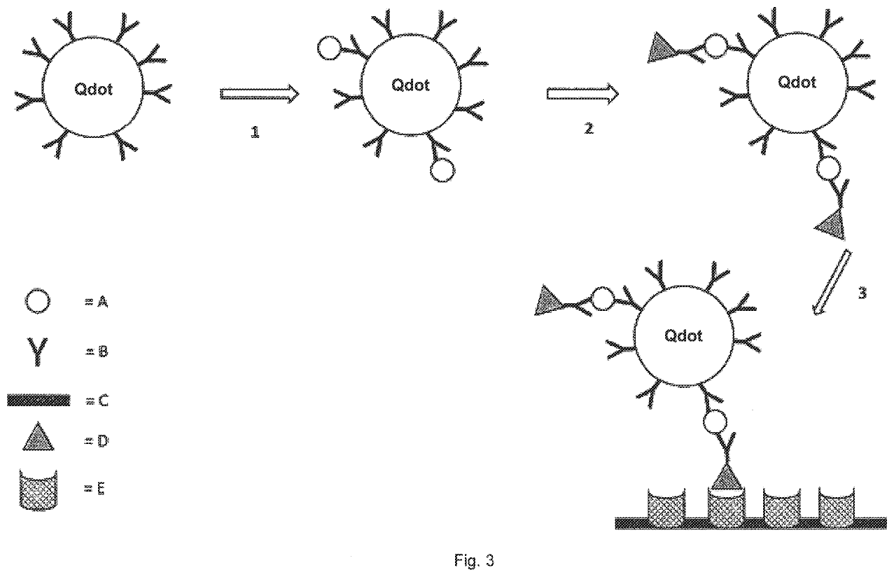

FIG. 3 shows a detection of bioindicators (A) in accordance with the invention using quantum dots (Qdots) which are coated with a capture molecule (B) for the bioindicator (A). The coated Qdots are added directly to the sample in step 1 and specifically bind the bioindicator (A) from the sample. Then, in step 2, a second capture molecule (B) is added, which is marked with a biotin molecule (D). The addition of Qdots and biotinylated capture antibodies (B-D) can also take place in reverse order. After incubation, this mixture of bound bioindicator Qdots is applied to the surface of a sample plate (C), for example a microtiter plate, in step 3. The surface of the sample plate (C) is coated with streptavidin (E). Components not bound to the surface of the sample plate (C) are removed by washing. The surface (C) is then evaluated by, for example, fluorescence microscopy, which detects the fluorescent Qdots bound to the surface of the sample plate (C).

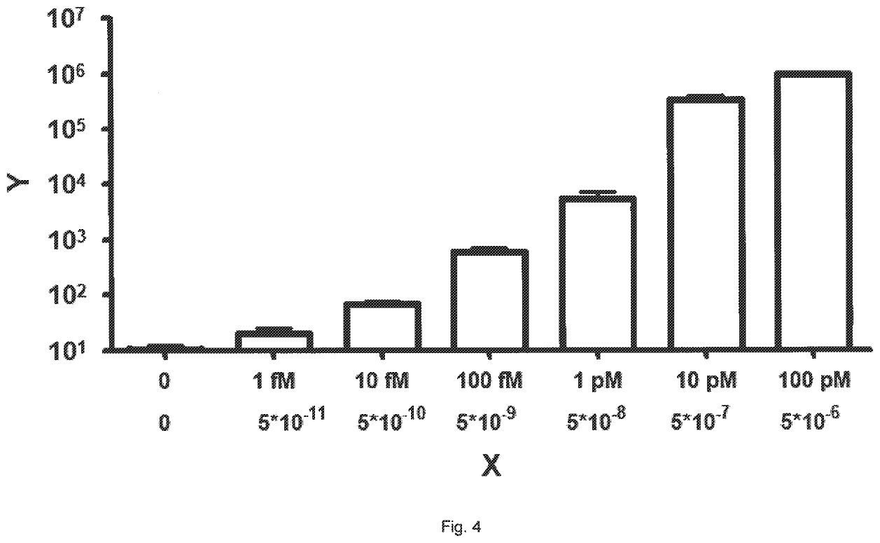

FIG. 4 shows a detection of streptavidin in different concentrations.

The different streptavidin concentrations are plotted on the abscissa (X-axis), indicated both as femtomolar (fM) to picomolar (pM) (upper number series) and in corresponding mg/ml (lower number series). The ordinate (Y-axis) indicates the numerical values of the detection signal, namely the TIRF microscope. The numerical values indicate the number of pixels detected.

According to FIG. 4, streptavidin can be detected in a decimal dilution series down to 1 femtomolar (fM) or 5*10⁻¹¹ mg/ml.

Fluorescence-labeled Alexa Fluor 633 streptavidin was used as a sample and diluted in saline tris(hydroxymethyl)aminomethane (TBS) at pH 7.4. The abovementioned dilution buffer TBS was used as negative control (0).

Commercial microtiter plates (Greiner Bio-one; Sensoplate Plus) with 384 reaction chambers (RC) and a glass bottom were used for the embodiment.

First, the surface of the microtiter plate was constructed or functionalized. For this purpose, 40 μl of 1% bovine serum albumin (BSA, Applichem) in phosphate-buffered saline (PBS, Sigma-Aldrich) were added to the RC of the microtiter plate and incubated at 4° C. overnight.

After incubation, the microtiter plate with the reaction chambers (RC) was washed three times with PBS. This was followed by the addition of 20 μl 1 mM EZ-Link NHS-PEG4-Biotin (ThermoScientific) as a capture molecule per RC and incubation for 30 min at room temperature (RT). After washing three times with PBS, the RC of the microtiter plate were each blocked with 40 μl 1% BSA in PBS, incubated for 1 h at RT and then washed five times with saline tris(hydroxymethyl)aminomethane (TBS, Serva) with 0.05% Tween-20 (Applichem) (TBS-T) and washed five times with saline tris(hydroxymethyl)aminomethane (TBS). The streptavidin sample, conjugated with Alexa Fluor 633 (ThermoScientific), was sequentially diluted in TBS and applied in quadruplicate to each 20 μl sample in the RC and incubated overnight at 4° C. After incubation, the RC was washed five times with TBS, admixed with 70 μl TBS with 0.03% ProClin (SigmaAldrich) per RC to prevent bacterial growth and sealed with a film. Since the sample itself is fluorescence-marked, it can be detected directly.

Spatially resolved microscopy was carried out in order to detect the monomeric proteins. The measurement was carried out in the TIRF microscope (Leica) with a 100-fold oil immersion objective. For this purpose, the glass bottom of the microtiter plate was coated abundantly with immersion oil, and the microtiter plate was introduced into the automated stage of the microscope. Then one image (1000×100 pixels) was recorded consecutively for each RC at 5×5 positions in the fluorescence channel Ex/Em=633/705 nm in order to obtain enough data points that the detection of individual protein molecules in front of the background signal was made possible. The maximum laser power (100%), an exposure time of 500 ms and a gain value of 1000 were selected. The image data were then analyzed. Intensity threshold values were set for each channel at 0.001% gray levels of the average negative control. Lastly, the number of pixels over all images in each RC was averaged and the mean values of the average pixel numbers of the replica values were then determined and the standard deviation specified.

The values are shown in FIG. 4. The results show a linear relationship between the concentration and the measurement signal over 6 log stages. With the method in accordance with the invention, the sample can be distinguished from the negative control up to a concentration of at least 1 fM or 5*10⁻¹¹ mg/ml.

This embodiment shows that the disadvantages of other methods, such as the low dynamic measuring range and maximum sensitivity only up to the picomolar range, can be overcome with the method in accordance with the invention. The values of this embodiment show a dynamic measurement range over 6 log stages and a sensitivity in the femtomolar range.

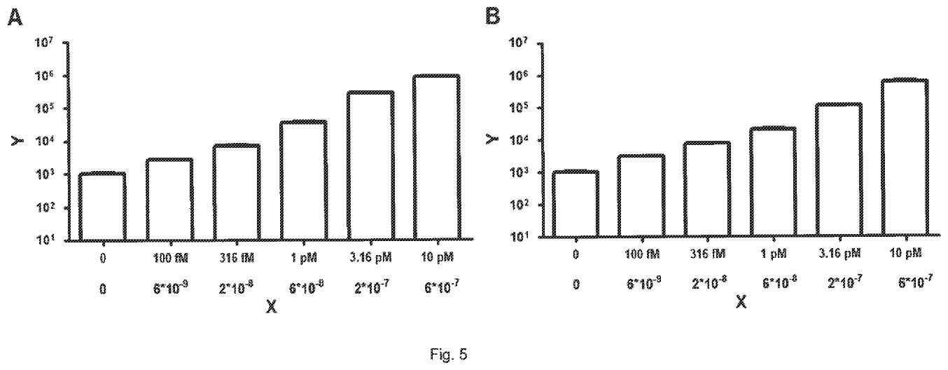

FIGS. 5A and B show detection of the monomeric protein construct 2ALFA2GFP in different concentrations, specifically in a dilution series of 10 pM or 6*10⁻⁷ mg/ml to 100 fM or 6*10⁻⁹ mg/ml. The different protein concentrations of the 2ALFA2GFP are plotted on the abscissa (X-axis), indicated both as femtomolar (fM) to picomolar (pM) (upper number series) and in corresponding mg/ml (lower number series). The ordinate (Y-axis) indicates the numerical values of the detection signal, namely the TIRF microscope. The numerical values indicate the number of pixels detected.

In this embodiment, the monomeric protein construct 2ALFA2GFP, which consists of two ALFA protein parts [11] and two GFP proteins (green fluorescent protein), was used as an example for the detection of a bioindicator. This construct was diluted in a dilution series of 10 picomolar (pM) to 100 femtomolar (fM) in saline tris(hydroxymethyl)aminomethane (TBS) at pH 7.4. The dilution buffer TBS was used as negative control (0). As a probe, the single domain antibody anti-ALFA, fluorescence conjugated with AlexaFluoro647 (FluoTag X2 anti-ALFA, nano tag) was used, which binds to the ALFA part of the 2ALFA2GFP protein construct.

For this protein construct, the ALFA tag and GFP protein were selected because they bind to their respective single domain antibodies with an affinity in the picomolar region.

Commercial microtiter plates with 384 reaction chambers (RC) and a glass bottom (Thermo Scientific) were used for the embodiment.

The anti-GFP single-domain antibody (GFP VHH, recombinant binding protein, gt-250, chromotek) was used as the capture molecule in a concentration of 5 μg/ml, which was diluted in TBS. 40 μl of this solution was added to each of the reaction chambers (RC) and incubated at 4° C. overnight. After incubation, the reaction chambers (RC) were washed five times with TBS-T and five times with TBS. Subsequently, the reaction chambers (RC) were each blocked with 80 μl 1% BSA in TBS, incubated for 1 h at room temperature (RT) and then washed five times with TBS-T and five times with TBS. The sample (protein construct 2ALFA2GFP) was sequentially diluted in TBS and, in quadruplicate, 20 μl of sample each was applied to the reaction chambers (RC) and incubated at 4° C. overnight. After incubation, the reaction chambers (RC) were washed five times with TBS. This was followed by the addition of the anti-ALFA-CF647 probe (20 μl) and incubation at RT for 1 h. Thereafter, the reaction chambers (RC) were washed five times with TBS, each mixed with 80 μl TBS with 0.03% ProClin (SigmaAldrich) in order to prevent bacterial growth and sealed with a film.

Spatially resolved microscopy was carried out in order to detect the monomeric proteins. The measurement was carried out in the TIRF microscope (Leica) with a 100-fold oil immersion objective. For this purpose, the glass bottom of the microtiter plate was coated abundantly with immersion oil, and the plate was introduced into the automated stage of the microscope. Then one image (1000×100 pixels) was recorded consecutively for each reaction chamber (RC) at 5×5 positions in two fluorescence channels (Ex/Em=633/715 nm and 488/525 nm) in order to obtain enough data points that the detection of individual protein molecules in front of the background signal was made possible. The maximum laser power (100%), an exposure time of 1000 ms and a gain value of 1300 were selected. The image data were then analyzed. Intensity threshold values were set for each channel at 0.001% gray levels of the average negative control in the corresponding channel. Lastly, the number of pixels over all images in each reaction chamber is averaged and the mean values of the average pixel numbers of the replica values are then determined and the standard deviation is specified.

The values are shown in FIG. 5. In FIG. 5A, the monomeric protein is detected directly via the fluorescence of GFP in channel 488; in FIG. 5 B the protein is detected via the anti-ALFA-AF647 probe in channel 633. The detection of the protein via the probe is comparable to the direct detection of the protein via the fluorescent GFP portion in its monomeric form. This comparison illustrates the sensitivity of the method.

The results show a linear relationship between the concentration of the protein construct used and the measurement signal. With this embodiment it can be shown that the method in accordance with the invention is suitable for detecting the protein construct in its monomeric form up to a concentration of 100 fM or 6*10⁻⁹ mg/ml and distinguishing it from the negative control. The values of this embodiment show a dynamic measurement range over 3 log stages and a sensitivity in the femtomolar range.

This embodiment also shows that the disadvantages of other methods, such as the low dynamic measuring range and maximum sensitivity only up to the picomolar range, can be overcome with the method in accordance with embodiments of the invention.

Literature source:
[11] Götzke H, et al. “The ALFA-tag is a highly versatile tool for nanobody-based bioscience applications”. Nat Commun. 2019 Sep. 27; 10(1):4403. doi: 10.1038/s41467-019-12301-7. PMID: 31562305; PMCID: PMC6764986.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A method for the quantitative and/or qualitative determination of bioindicators, said method comprising the following steps:
a) immobilizing capture molecules for the bioindicators on a substrate,
b) bringing the bioindicators of a sample into contact with the capture molecules,
c) immobilizing the bioindicators on the substrate by binding to capture molecules,
d) bringing the bioindicators into contact with probes containing at least one detection molecule,
e) removing non-specifically bound molecules and particles, and
f) binding the probes to the bioindicators,
wherein the probes are capable of emitting a specific detection signal and steps b) and d) can take place simultaneously or d) before b), and wherein probes and capture molecules are used which have affine molecules or molecule parts that bind to at least one specific binding site of the bioindicators and these affine molecules or molecule parts of the probes and capture molecules do not overlap one another.

2. The method according to claim 1, wherein the affine molecules or molecule parts of the probes and/or capture molecules bind to at least two specific binding sites of the bioindicators.

3. The method according to claim 1,
wherein the affine molecules or molecule parts of the probes and/or capture molecules bind to at least two specific binding sites of the bioindicators, wherein these binding sites of the probes and/or capture molecules bind to at least two identical binding sites of a bioindicator and/or bind to at least two different binding sites of at least two different bioindicators.

4. The method according to the claim 3,
wherein
by bringing the bioindicators into contact with the capture molecules, said bioindicators are immobilized on the substrate by binding to the capture molecules.

5. The method according to claim 1,
wherein
after bringing the bioindicators into contact with the probes, non-specifically bound molecules and particles are removed by washing.

6. The method according to claim 1,
wherein
probes which bind to the bioindicators are selected, wherein the probes are also capable of emitting a specific signal.

7. The method according to claim 1,
wherein
the detection molecules of the probes comprise fluorochromes and/or fluorescent proteins.

8. The method according to claim 1,
wherein
the bringing of the bioindicators into contact with the capture molecules and the probes takes place simultaneously.

9. The method according to claim 1,
wherein
the bringing of the bioindicators into contact with the probes takes place prior to bringing the bioindicators into contact with the capture molecules.

10. The method according to claim 1,
wherein
the sample is fixed prior to binding the probes to the bioindicators.

11. The method according to claim 1,
wherein
quantum dots are used instead of probes, which quantum dots are coated with capture molecules having affine molecules or molecule parts which bind to at least one specific binding site of the bioindicators.

12. The method according to claim 1,
wherein
the sample is treated with detergents.

13. The method according to claim 1,
wherein
a spatially resolved determination of the probe signal takes place.

14. The method according to claim 1,
wherein
the substrate comprises plastic, silicon or silicon dioxide, and/or glass.

15. The method according to claim 1,
wherein
the substrate has a hydrophilic surface prior to immobilizing capture molecules on the substrate.

16. The method according to claim 15,
wherein
the hydrophilic layer is selected from the group comprising or consisting of PEG, poly-lysine and dextran, or derivatives thereof.

17. The method according to claim 1,
wherein
functionalization with amino groups is effected by bringing the substrate into contact with APTES (3-aminopropyl-trietoxy silane) or ethanolamine.

18. The method according to claim 1,
wherein
bringing the substrate into contact with APTES (3-aminopropyl-trietoxy silane) takes place in the gas phase.

19. The method according to claim 1,
wherein
the capture molecules are covalently bonded to the substrate or to the coating.

20. The method according to claim 1,
wherein
the binding sites of the bioindicators are multispecific epitopes and the affine molecules or molecule parts of the capture molecules and/or probes are multispecific antibodies or parts thereof.

21. The method according to claim 1,
wherein
the probes are marked with fluorescent dyes.

22. The method according to claim 1,
wherein
detection takes place by spatially resolving fluorescence microscopy.

23. A method for detecting a disease, comprising the method of claim 1.

24. A method for monitoring therapies with bioindicators and/or checking the effectiveness of active substances and/or therapeutic methods or determining if a person is to be included in a clinical study, comprising the method of claim 1.

25. A kit for carrying out the method according to claim 1, wherein the kit comprises:
substrate,
capture molecules; and
probe molecules,

26. A kit for carrying out the method according to claim 1, , wherein the kit
substrate,
capture molecules; and
Qdots, wherein the Qdots are coated with capture molecules having affine molecules or molecule parts which bind to at least one specific binding site of the bioindicators.
